(19) United States
(12) Patent Application Publication
(10) Pub. No.: US 2009/0149522 A1
(43) Pub. Date: Jun. 11, 2009

(54) Method of preserving a customised composition

(76) Inventors: Geetha Basappa, Sharnbrook (GB); Maria Teresa Belmar, Sharnbrook (GB); Ana Martin-Herranz, Madrid (ES); Iain Sunil Young, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/989,148

(22) PCT Filed: Jul. 28, 2006
(86) PCT No.: PCT/EP2006/007595

§ 371 Date: Jul. 7, 2008

(30) Foreign Application Priority Data
Jul. 30, 2005 (EP) 05254801.3

(51) Int. Cl. A01N 43/50; A61K 8/36; A01N 37/06; A01N 33/12; A01P 3/00; A61Q 19/00; A01P 1/00; A01N 31/14; A01N 37/10; A01N 37/02

(52) U.S. Cl. 514/390; 514/558; 514/549; 514/532; 514/546; 514/389; 514/642; 514/723

(57) ABSTRACT

The invention concerns a process for producing a preserved customised consumer composition, preferably from a Vending machine, wherein a plurality of ingredient streams are mixed together in predefined relative amounts, characterised in that At least two streams consist of water and preservative only, at least two of which having different concentrations of preservative. The Invention permits the final preservative mix to be independently variable to the formation.

### BACKGROUND AND PRIOR ART

The present invention relates to a process for preserving instant compositions, such as food products or personal care products such as skin creams or lotions.

Machines and processes which can manufacture consumer products at the point-of-sale or point-of-use are well-known in the art. The advantage of such processes is primarily flexibility in delivering a product which is suited to the consumer as opposed to having to purchase a one-size-fits-all off-the-shelf version. However, there are considerable technical challenges involved in making these processes work in practice.

An example of such a machine for making personalised skin creams is found in WO-A-03/045347 or WO-A-03/045346 which teach that a personalised skin cream can be formed by blending together an anhydrous base material of high melting point fatty acid and nonionic surfactant together with heated water.

Consumer products such as skin creams or food products may need to be preserved to prevent the growth of bacteria, fungus, yeast, molds, etc. However, the amount of preservative must not be so high that it is above a toxicology limit. In other words a minimum limit is imposed for hygiene purposes, and an upper limit from a toxicology viewpoint. This can result in a narrow preservation window within which to operate.

Additionally, if the final product is also to be preserved then each ingredient stream must also be individually preserved so that they do not go off on storage before mixing takes place.

Therefore, a customised consumer product, which by definition, can have a wide range of formulations, will have a final preservative level which is dependent upon the preservative level in the individual streams from which it is made. Ideally the preservative level of the final product would be independently variable to the formulation.

Therefore, the problem to be solved is how to provide a process which can give a predetermined amount of preservative in the consumer product whilst retaining full formulation flexibility.

For some consumer products, there is an identifiable ‘base’ stream which is always present in the final product at a high amount, and thus the final level of preservative can to a large extent be governed by the preservative level in the ‘base’ stream. However, even in this situation, the final level of preservative is not independently variable to the formulation. This problem is particularly acute for skin creams or lotions because they do not have an identifiable base stream.

Another difficulty specific to personalised skin creams is the fact that some streams are aqueous and some are oil based. Therefore, the choice of preservative for each stream is further constrained by the solubility of the preservative. Often the oil-based streams do not have any preservative in them because it is not necessary and oil-soluble preservatives are rare. Therefore a final product rich in oil-based ingredient streams may be low in preservative.

Surprisingly, the present inventors have solved this problem by providing two streams which comprise water and preservative only. In addition the two streams have a different effective concentration of preservative, so that a final target preservative level can be achieved by relative dosing of the two preserved water streams.

WO-A-98/30189 discloses an apparatus for formulating a point-of-sale cosmetic composition. Separate containers are filled with components of base compositions, one possible component being a preservative (not necessarily one component per receptacle). Additional receptacles contain optional additive materials. Streams from these receptacles are mixed to create the product. There is no suggestion of having at least two receptacles each containing only water and a preservative.

### DEFINITION OF THE INVENTION

Thus, the present invention provides a process for producing a preserved customised consumer composition, wherein a plurality of ingredient streams are mixed together in predefined relative amounts, characterised in that at least two streams consist of water and preservative only, at least two of which having different concentrations of preservative, the ratio of preservative levels between the two streams preferably being from 1.5:1 to 50:1.

### DETAILED DESCRIPTION OF THE INVENTION

### Customised Consumer Product

The present invention is applicable to any point-of-sale customised manufacturing process which involves blending together separate component ingredients to produce the final customised product. For example, such product types as food sauces, hair shampoos and conditioners, skin creams, liquid detergents, toothpastes, beverages, ice cream etc. The products of the invention comprise water.

Such a process preferably takes place at a temperature of below 50° C., more preferably at room temperature.

The process is usually carried out inside a vending machine, which contains within a plurality of ingredient streams feeding an ingredient source to a mixing stage.

### Skin Creams and Lotions

The present invention is particularly useful for customised skin creams and lotions.

Cosmetic compositions of the present invention may be in any form. These forms may include emulsified systems such as lotions and creams, microemulsions, roll-on formulations, mousses, ointments (hydrophilic and hydrophobic), aerosol and non-aerosol sprays and pad-applied formulations.

Resulting personal cosmetic compositions according to the invention may typically contain 50% to 90% water, more preferably 70% to 85% water, and sufficient fatty acid material and nonionic surfactant to provide the desired structure. Suitable levels of fatty acid are generally in the range 5% to 25%, more preferably 10% to 20%, and suitable levels of nonionic surfactant are generally in the range 0.5% to 10%, more preferably 2% to 8%.

### Preservatives

Preservatives are any ingredients which prevent the build up of micro-organisms such as bacteria, fungus, yeast, molds etc. Well-known preservatives suitable for use in the present invention include sorbates, benzoates, para-hydroxy benzoates, sulphites, nitrates, acetates. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients. Preservatives are preferably employed in amounts ranging from 0.01% to 2% by weight of the composition.

Some preservatives are more effective as an antibacterial and others as an anti-fungal. Such preservatives are herein defined as antibacterial and antifungal preservatives respectively.

In general, preservatives are only necessary from a hygiene perspective if the water activity of a stream or product is greater than 0.7. Antibacterial preservative is generally only necessary if the water activity is greater than 0.9.

A particularly preferred antibacterial preservative is DMDM hydantoin (Trade name: Glydant Ltd) supplied by Lonza. A particularly preferred antifungal preservative is sodium methyl parabens (Trade name: Nipagin M sodium) and/or sodium propyl parabens (Trade name: Nipasol M sodium), supplied by Clariant.

Preferably, the ratio of the effective preservative levels between the two water streams is from 1.5:1 to 50:1, more preferably from 2:1 to 20:1, and most preferably from 2.5:1 to 10:1. By ‘level’ is meant amount by weight.

In a preferred class of embodiments, more than one kind of preservative is in each of the water streams and the ratio of the first to the second preservative is the same for the two streams.

It can also be advantageous if at least two of the streams having different concentrations of preservative comprise both an antifungal preservative and an antibacterial preservative. In that case, it is preferable if the ratio of antifungal to antibacterial in the first stream is substantially the same as the ratio of antifungal to antibacterial in the second stream.

### Skin Cream Ingredients

A preferred product type for the present invention is skin creams or lotions.

The ingredients of such skin creams have an effect on moisturisation degree, sun protection factor, water resistance or a combination thereof. The primary functionality of personal care products according to the invention is generally in their moisturisation capacity. Other functionalities are anti-ageing and effect on firmness.

Generally the functionality of a personal care composition may be controlled by the level and variation of emollients and humectants.

### Emollients

Emollients are typically oil based and in liquid form and may be selected from the group comprising hydrocarbons, silicones, synthetic esters or vegetable esters or their combinations.

Silicone oils may be divided into the volatile and non-volatile variety. The term “volatile” as used herein refers to those materials, which have a measurable vapour pressure at ambient temperature. Volatile silicone oils are preferably chosen from cyclic or linear polydimethyl siloxanes containing from 3 to 9, preferably from 4 to 5 silicon atoms. Linear volatile silicone materials generally have viscosities less than about 5 centistokes at 25° C., whilst cyclic materials typically have viscosities of less than about 10 centistokes. Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile silicone emollients useful in the present compositions are the polydimethyl siloxanes having viscosities from about 10 to about 400 centistokes at 25° C.

Among the ester emollients are:
(1) Alkenyl or alkyl esters of fatty acids having 10 to 20 carbon atoms. Examples thereof include isoarachidyl neopentanoate, isononyl isononanoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyo behenate.
(5) Sterol esters of which cholesterol fatty acid esters are examples thereof.

Preferred emollients are selected from the group comprising isoparraffins, mineral oil, cyclic or linear polydimethylsiloxanes, vegetable based oils such as sunflower oil and olive oil or a combination of any of these.

The most preferred esters are isoarachidyl neopentanoate and isononyl isononanoate.

Fatty acids having from 10 to 30 carbon atoms may also be included for compositions of this invention. Illustrative of this category are pelargonic, lauric, myristic, palmitic, stearic, isostearic, hydroxystearic, oleic, linoleic, ricinoleic, arachidic, behenic and erucic acids.

### Humectants

Humectants may also be employed in compositions of this invention. The humectant aids in increasing the effectiveness of the emollient, reduces scaling, stimulates removal of built-up scale and improves skin feel.

Examples of humectants are glycerol(glycerine), oils, and other alcohols. Typical Polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3butylene glycol, 1,2,6-hexanetriol, ethoxylated glycerol, propoxylated glycerol and mixtures thereof. The amount of humectant may range anywhere from 0.5 to 30%, preferably between 1 and 15% by weight of the final personal care composition.

### Thickener

Products of the present invention beneficially comprise additional thickener. Suitable thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Alternative thickener is day. Amounts of the thickener may range from 0.0001 to 2%, usually from 0.001 to 1%, by weight of the personal care composition, if at all.

Collectively the water, solvents, silicones, esters, fatty acids, humectants and/or thickeners will constitute the composition in amounts from 1 to 99.9%, preferably from 80 to 99% by weight.

### Surfactants

Surfactants may also be present in cosmetic compositions of the present invention. Total concentration of the surfactant will range from 0.1 to 40%, preferably from 1 to 20%, optimally from 1 to 5% by weight of the composition. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C8-C20 acyl isethionates, acyl glutamates, C8-C20 alkyl ether phosphates and combinations thereof.

### Minors

Optionally, other minor ingredients may be added.

Actives are defined as skin benefit agents other than emollients and other than ingredients that merely improve the physical characteristics of the composition. Although not limited to this category, general examples include additional anti-sebum ingredients such as talcs and silicas, and sunscreens. Further examples include silk protein, fragrances, colouring agents, healthy skin ingredients such as AHA, collagen, amino acids; vitamins such as vitamin A and vitamin E, triple lipids such as lecithin, soy sterol; or combinations thereof.

Preferred sunscreens are such materials as ethylhexyl p-methoxycinnamate, available as Parsol MCX, and benzophenone-3, also known as Oxybenzone. Inorganic sunscreen actives may be employed such as microfine titanium dioxide, polyethylene and various other polymers. Amounts of the sunscreen agents will generally range from 0.1 to 30%, preferably from 2 to 20%, optimally from 4 to 10% by weight.

Compositions of the present invention may also contain water-soluble vitamins. The term water-soluble defines substances with a solubility of at least 0.1%, preferably at least 1%, optimally at least 5% by weight in water. Illustrative water-soluble vitamins are Niacin, Vitamin B6, Vitamin B6, Vitamin C and Biotin. One source for Vitamin C is a product sold under the trademark of Vitazyme C available from the Brooks Company. Niacin, Vitamin B and Biotin are available from Roche Pharmaceuticals. Total amount of vitamins in compositions according to the present invention may range from 0.001 to 1%, preferably from 0.01 to 0.6, optimally from 0.1 to 0.5% by weight.

Keratolytic agents such as C2-C25 α-hydroxy alkanoic acids may also be incorporated into compositions of this invention. Illustrative of this group of materials are glycolic, lactic, α-hydroxyoctanoic acids and salts thereof. The salts may be selected from alkalimetal, ammonium and C1-C20 alkyl or alkanolammonium counterions. Levels of α-hydroxyalkanoic acids may range from 0.001 to 10%, preferably between 0.2 and 1%, optimally between 0.4 and 0.5% by weight.

Minor adjunct ingredients may also be present in the cosmetic compositions. Among them may be the water-insoluble vitamins such as Vitamin A Palmitate, Vitamin E Acetate and DL-panthenol.

Another adjunct ingredient can be that of an enzyme. Particularly preferred is superoxide dismutase, commercially available as Biocell SOD from the Brooks Company, USA.

Natural vegetable materials from renewable resources are often desirable in cosmetic compositions. For instance, cosmetic compositions of the present invention may include β-glucan derived from oats, commercially available under the trademark Microat SF from Nurture Inc., Missoula, Mont.

Colorants, fragrances, opacifiers and abrasives may also be included in compositions of the present invention. Each of these substances may range from about 0.05 to about 5%, preferably between 0.1 and 3% by weight.

### EXAMPLES

A machine was build which has 25 skin cream ingredients, each stored separately in the machine. Each ingredient store is connected to an in-line mixer unit via its own peristaltic pump. When it is desired to make a personalised skin cream, the formulation is first determined. Once determined, a computer unit controls the flow rates of each of the pumps in the 25 lines to bring the ingredients together in the in-line mixer to produce a single homogenous skin cream. The skin cream can be made in incremental batches of 50 g.

Two of the ingredient streams are purely water and preservative. They have the following composition:

| Ingredient | Stream 1 | Stream 2 |
|---|---|---|
| Glydant Ltd liquid | 0.3 | 1.05 |
| Na Methyl parabens | 0.2 | 0.7 |
| Na Propyl parabens | 0.1 | 0.35 |
| Water | 99.5 | 97.9 |

### Calculation for the Ratio of Streams 1 and 2

The following formulation is to be prepared:

| Ingredient | Wt % |
|---|---|
| Surfactant base | 33 |
| Glycerine 99.5% | 2 |
| DC345 | 5 |
| Catiol | 2 |
| Myritol | 2 |
| Water (additional) | 56 |
| Total | 100 |

Each stream which contains preservative, has the three preservatives in the same ratio: 2:1:3.

The total amount of preservative, assuming no preservative in the water stream is 0.15%, which is below the minimum hygiene limit. The target preservative level is 0.61%. Therefore 0.3% more preservative must come from the water streams.

If:

Water from stream 1=x

Water from stream 2=y

Then:

x+y=56 (1)

Additionally:

0.006x+0.021y=0.3 (2)

Solving equations (1) and (2) gives:

x=51.6%

y=4.4%

Therefore, of the 56% which is water, 51.6% comes from the water stream which is low in preservative and 4.4% from the stream high in preservative.

If there was only one preserved water stream, the amount of preservative in the made skin cream would be uncontrolled and could be below the minimum hygiene limit or above the maximum toxicology limit. In either case, the skin cream would not be suitable for commercial use.

1. A process for producing a preserved customised consumer composition, wherein a plurality of ingredient streams are mixed together in predefined relative amounts, characterised in that at least two streams consist of water and preservative only, at least two of which having different concentrations of preservative, the ratio of preservative levels between the two water streams preferably being from 1.5:1 to 50:1.

2. A process according to any preceding claim, wherein the ratio of the effective preservative levels between the two water streams is from 2:1 to 20:1, preferably from 2.5:1 to 10:1.

3. A process according to any preceding claim, wherein more than one kind of preservative is in each of the water streams and the ratio of the first to the second preservative is the same for the two streams.

4. A process according to any preceding claim, wherein at least two of the streams having different concentrations of preservative comprise both an antifungal preservative and an antibacterial preservative.

5. A process according to claim 4, wherein the ratio of antifungal to antibacterial in the first stream is substantially the same as the ratio of antifungal to antibacterial in the second stream.

6. A process according to any preceding claim, wherein the preservative comprises one or more preservative compounds selected from sorbates, benzoates, para-hydroxy benzoates, sulphites, nitrates, acetates, alkyl esters of para-hydroxybenzoic acid, hydantoin derivatives such as DMDM hydantoin, propionate salts and quaternary ammonium compounds, for example, phenoxyethanol, salts of methyl parabens, propyl parabens, imidazolidinyl urea, sodium dehydroacetate and benzyl alcohol.

7. A process according to any preceding claim, wherein the preserved composition contains from 0.01% to 2% by weight of preservative.

8. A process according to any preceding claim, wherein at least one ingredient stream and/or the preserved composition has a water activity greater than 0.7, preferably greater than 0.9.

9. A process according to any preceding claim, wherein the preserved composition is a pesonalised cosmetic composition.

10. A process according to claim 6, wherein the cosmetic composition is a skin cream or lotion.
